(19) United States
(12) Patent Application Publication
(10) Pub. No.: US 2008/0038233 A1
(43) Pub. Date: Feb. 14, 2008

(54) Treatment of Spinal Conditions

(75) Inventors: Anthony John Freemont, Manchester (GB); Judith A. Hoyland, Manchester (GB); Christine Le-Maitre, Manchester (GB); Stephen Richardson, Manchester (GB)

Correspondence Address: DAVIS WRIGHT TREMAINE LLP/Los Angeles, 865 FIGUEROA STREET, SUITE 2400, LOS ANGELES, CA, 90017-2566, US

(73) Assignee: The University of Manchester, Manchester (GB)

(21) Appl. No.: 10/590,508

(22) PCT Filed: Feb. 25, 2005
(86) PCT No.: PCT/GB05/00713

§ 371 Date: Nov. 30, 2006

(30) Foreign Application Priority Data
Mar. 2, 2004 (GB) 0404656.1

(51) Int. Cl. A61K 35/00; A61F 2/44; A61P 19/04; C12N 5/06

(52) U.S. Cl. 424/93.21; 435/372; 435/377; 623/17.16

(57) ABSTRACT

The invention relates to isolated mesenchymal stromal stem cell (MSSC) that has been differentiated in vitro towards, or to, an intervertebral disc (IVD) cell phenotype for use as medicaments. The cells may be used to treat spinal conditions characterized by degeneration of the intervertebral disc. The cells may also be genetically transformed with an exogenous gene coding for a protein that reduces degeneration of an intervertebral disc.

The present invention relates to the treatment of spinal conditions characterized by a degeneration of the intervertebral disc (DIVD).

DIVD is believed to be caused by a loss or change in matrix production of the intervertebral disc (IVD). DIVD may arise as a consequence of age-related changes of the IVD, spondylolysis etc.

One example of a spinal condition according to the invention is low back pain (LBP) LBP is multifactorial. However it is believed that DIVD is a very common cause of LBP. About 11 million people in the UK experience LBP for at least 1 week each month; it leads to considerable loss of working days (108 million in 2000, and an estimated £11 billion in lost production). It also impacts significantly on the National Health Service (e.g. >6 million clinical consultations/annum) and social services. Although there are many causes of LBP, the role of DIVD in LBP is becoming clearer. Imaging studies indicate a link between DIVD per se and LBP. Furthermore the inventors of the application in suit have shown how they are linked mechanistically through nociceptive nerve ingrowth into degenerate IVD. The most clinically significant correlations however, are between IVD space narrowing due to DIVD and chronic LBP (CLBP), particularly when the former is associated with abnormal alignment and movement of the elements of the spinal motion segment. IVD space narrowing develops as DIVD progresses. Since it is now possible, using MR imaging, to recognise early stages of DIVD (i.e. before narrowing of the IVD space occurs) the combination of early diagnosis of DIVD and an effective treatment to halt/reverse its progression raises the prospect of significantly decreasing the incidence of CLBP. Furthermore novel forms of tissue engineering offer an approach to treating more advanced stages of DIVD.

The IVD provides reversible resistance to compressive, rotational and tensile loads applied to the vertebral column. These properties are a function of the structure of its major components—inner nucleus pulposus (NP), outer annulus fibrosus (AF) and superior and inferior cartilaginous end plates (CEP). These are, in general, hypocellular and avascular tissues, composed mainly of collagens and proteoglycans. The cells that populate these tissues maintain tissue composition and integrity. Unlike many tissues in which cells are in direct contact with one another, within the IVD, the cells are widely separated, and have an intimate two-way dynamic relationship with the matrix. In vivo IVD cells not only manufacture and maintain the matrix, but receive all nutrients by diffusion and bulk flow across it and through their interactions with it obtain key regulatory information, particularly that relating to the physical environment.

Many attempts have been made to develop therapies that will prevent the development of, and/or treat, spinal conditions characterized by degeneration of the intervertebral disc. Three avenues of research relate to:
1) The development of gene therapy techniques for delivering biologically active molecules to reverse the molecular processes leading to degeneration of the IVD.
2) The introduction of conditioned cells to produce normal IVD tissue to replace that lost in the process of IVD degeneration.
3) The production of tissue engineered IVD implants.

Each of these avenues requires the use of suitable cells. However, to date no useful source of cells exist and it is one object of the present invention to provide methods for the production and transformation of cells for use in 1-3 above.

According to a first aspect of the present invention there is provided an isolated mesenchymal stromal stem cell (MSSC) that has been differentiated in vitro towards, or to, an IVD cell phenotype for use as a medicament.

According to a second aspect of the present invention there is provided the use of an isolated mesenchymal stromal stem cell (MSSC) that has been differentiated in vitro towards, or to, an IVD cell phenotype in the manufacture of a medicament for the treatment of spinal conditions characterized by degeneration of the intervertebral disc.

According to a third aspect of the present invention there is provided a method of treating spinal conditions characterized by degeneration of the intervertebral disc comprising administering to a diseased intervertebral disc of a subject in need of such treatment an isolated mesenchymal stromal stem cell (MSSC) that has been differentiated in vitro towards, or to, an IVD cell phenotype.

By the term “IVD cells” we mean the cells that populate the inner nucleus pulposus (NP), outer annulus fibrosus (AF) and superior and inferior cartilaginous end plates (CEP) of the IVD. The cells within these three areas are also referred to herein as NP cells, AF cells or CEP cells respectively. Collectively these cells are referred to as IVD cells.

By the term “differentiated in vitro towards, or to, an IVD cell phenotype” we mean the MSSC cells have been treated such that the stem cell has matured down the lineage of NP cells, AF cells or CEP cells. Furthermore the morphology and particularly the functionality of the MSSC cells has changed to be more like NP cells, AF cells or CEP cells. It will be appreciated that it is preferred that the differentiation is complete as possible. However incomplete differentiation down the IVD lineage may still result in cells useful according to the present invention.

It is preferred that the cells differentiated in vitro towards, or to, an IVD cell have a phenotype that is distinguishable from chondrocytes (or precursors of chondrocyte lineage) in articular cartilage or other forms of cartilage. Accordingly differentiation procedures employed according to the present invention preferably ensure that the resulting NP, AF or CEP cell is not a chondrocyte. One way by which a skilled person can differentiate between cells differentiated towards, or to, an IVD cell phenotype and cells differentiated towards, or to, chondrocytes is to examine the extracellular matrix produced by each type of cell. When chondrocytes are transplanted into the nucleus pulposus of rabbit intervertebral discs it has been found that the matrix formed is more solid than the normal gel like matrix of the nucleus pulposus and also has a different composition. Accordingly a skilled person can analyse the matrix produced by a cell and will be able to identify whether a cell is of an IVD or chondrocyte phenotype. Thus the suitability of a differentiation step for use according to the invention (see below) may preferably be assessed by ensuring that the cells produced thereby produce a correct matrix (i.e. a IVD matrix rather than a chondrocyte matrix). In this respect a skilled person will appreciate that an IVD matrix will be characterised by at least one, and preferably each of, the following:
a) aggrecan gene expression should be greater than collagen type II gene expression;
b) the proteoglycan versican should be expressed; and
c) the GAG: hydroxproline ratio (i.e proteoglycan:collagen ratio) should be greater than 10:1.

The present invention is based upon research relating to the IVD conducted by the inventors. They have realized that a key factor in the repair and regeneration of IVD tissue is to ensure that there are suitable cells in the IVD to mediate the repair/regeneration.

It has previously been proposed that suitable cells may be injected into the IVD and thereby regenerate the NP. One approach has been to harvest mature IVD cells from a patient's IVD and re-introduce them into an injured tissue. For instance, DE 42 19 626 contemplates the removal of somatic IVD cells from the spine and then reintroducing the cells (following genetic manipulation to improve their reparative qualities). However the inventors have found, to their surprise, when they explored the biology of these somatic cells in more detail, that this approach is flawed. This is because they have discovered that cells from a patient's damaged IVD have a senescent phenotype and thereby function poorly and rapidly die. Furthermore when such harvested cells are infected with a therapeutic gene, and injected into explant IVD, the inventors found that the cells only poorly express the introduced gene. The inventors also investigated cell replication potential (which falls with senescence) and expression of the cyclin-dependent kinase inhibitor P16^{INK4A} protein (upregulated during cellular senescence), in tissue and cells of normal and degenerate NP. This showed age-related: decrease in replication potential; and increase in cellular expression of P16^{INK4A}; in the normal NP. NP cells of degenerate IVD, however, even from young patients, exhibited a decrease in replicative potential and increased expression of P16^{INK4A} equivalent to those of normal individuals who were as much as 40 years older. Furthermore, real time RT PCR demonstrated that there is a direct relationship between expression of senescent biomarkers and the genes for: the matrix degrading enzymes MMPs 3 & 13; the aggrecanase, ADAMTs 5; and type I collagen; and an inverse relationship with expression of type II collagen and aggrecan.

The inventors realized that such data suggest that degenerate NP cells show a senescent phenotype with an altered cellular metabolism that may prevent their subsequent use in repairing the IVD. It was therefore realized that the use of cells as proposed by the prior art (e.g. DE 42 19 626) was unsuitable for the treatment of spinal conditions.

Having established that senescence was a problem in the use of cells for the treatment of spinal conditions, the inventors exerted inventive endeavor in attempt to identify other cell types that may be useful in the modulation of spinal conditions such as low back pain.

As described in more detail below, and in the Example, the inventors established that cells according to the first aspect of the invention may be used to treat spinal conditions. The use of cells according to the invention would not have been contemplated by the skilled person. In fact their use would be considered to be counter-intuitive. This is because the prior art suggests that cells taken directly from an IVD would be the best source of cells for use in therapy. Such a source of cells would immediately be selected because (a) the skilled person would have no knowledge of the senescence problems associated with IVD cells from diseased IVDs; and (b) the skilled person would be attracted to use differentiated IVD cells because they are already of the correct phenotype for possible manipulation and reintroduction into a diseased IVD.

It is possible that a skilled person may consider isolating IVD cells from a healthy IVD and transforming such cells prior to injection into a diseased IVD. The selection of such cells may have fewer problems with respect to senescence. However the choice of these cells would not be considered by a clinician because the extraction of such cells from a healthy IVD would result in an unacceptable risk that the patient may suffer as a consequence of the procedure and the healthy IVD would be damaged and thereby exacerbate the spinal condition.

Furthermore there would also be a technical prejudice against the selection of mesenchymal stromal stem cells for use in therapy of spinal conditions. Stem cells per se would not be useful and, to date, there have been no known methods for the satisfactory differentiation of MSSCs into IVD cells. Accordingly the skilled person, who would not benefit from the inventors knowledge relating to senescence or their skills in differentiation of MSSC, would automatically consider the use of cells that are already differentiated into IVD cells. The differentiation techniques used to develop cells according to the invention represent further important aspects of the present invention and are discussed in more detail below.

Any form of mesenchymal stromal stem cell may be used according to the invention. Such cells may be harvested from blood, bone marrow, or adipose tissue (autologous MSSCs) according to techniques known to the art.

It is preferred that the MSSCs are taken from bone marrow (e.g. form the sternum, femur or iliac crest). A most preferred method of harvesting such cells is described in method 1.1.1 of Example 1.

It will be appreciated that, as an alternative to harvesting stem cells, that stem cell lines grown in vitro may be used as a source of cells for differentiation.

The inventors have established that a number of differentiating techniques may be used (singularly or in combination) to cause mesenchymal stromal stem cells to differentiate towards IVD cells. Methods that may be employed according to the invention include:

(A) IVD cell induction medium—The inventors have established that standard cell culture media may be manipulated such that it includes active ingredients that promote differentiation of MSSCs into IVD cells.

NP cells initially share a common phenotypic lineage with articular chondrocytes and can advance along the common lineage pathway by growing them in a medium enriched with growth factors such as TGFβ. TGFβ usage does not usually advance MSSC beyond the common precursor of the articular chondrocyte/NP cell. Therefore, for differentiation of cells that are distinguishably of an IVD phenotype, it is preferred that this differentiation step is combined with other steps (see below).

By way of example, TGF-β3 may be added to cell culture media to promote differentiation. A preferred IVD cell induction medium comprises DMEM/HAMs F12 1:1 supplemented with glucose, L-ascorbic acid, suitable antibiotics, TGF-β3, dexamethosone, sodium pyruvate, proline and ITS+1 premix (ITS mix from Sigma with a premix comprising 1 mg/ml insulin, 0.55 mg/ml transferin, 0.5 μg/ml sodium selinite; 50 mg/ml BSA and 470 μg/ml linoleic acid). The cells may be cultured in the media for at least 1 day, preferably at least 5 days and most preferably for about 12 days. A most preferred IVD cell induction medium is disclosed at 1.1.3.1 of Example 1.

In addition to TGF-β in the media, other growth factors have a similar or greater effect in inducing an NP phenotype, particularly in increasing the amount of proteoglycan expressed/produced. One such growth factor is cartilage derived growth factor (CDMP) 1 and 2. The inventors have shown that CDMP at a concentration of 10-100 ng/ml can induce MSSC differentiation to an NP phenotype with a substantial increase in proteoglycans (see Example 5).

(B) Gel encapsulation—Encapsulation of stem cells in alginate or other gels facilities differentiation. Encapsulation in this or other gels may be used to promote differentiation and is particularly useful when used in conjunction with another technique (e.g. IVD cell induction medium). When this is the case the encapsulated cells may be exposed to the media for at least 1 day, preferably at least 5 days; more preferably for at least 12 days; and for periods up to 35 days.

A preferred differentiation technique utilising encapsulation is described at 1.1.3.2 and 1.1.3.3 of Example 1.

An alternative, and most preferred encapsulation method, comprises resuspending cells in 1.2% medium viscosity alginate in 0.15M NaCl at a density of about 5×10⁶ cells/ml and then polymerising the alginate to form a layer.

(C) Application of Load—During their studies the inventors discovered that degenerate NP cells appear to be hypersensitive to loading at pressures as low as 1 psi (0.0069 Mpa [the minimum load on the normal intervertebral disc in vivo is 0.3 Mpa]). Experiments also showed that cellular responses were dependent on cell type and the frequency and amount of load applied. For instance significant differences were observed in gene expression from degenerate NP cells loaded at 5 psi (0.0345 MPa) and at 7 psi (0.0483 MPa).

Finding this lead them to realize that cells, useful in the treatment of back pain might be generated by applying particular loading conditions to cells during their preparation. They have subsequently shown that loading MSSC induces an NP phenotype. Load may be exerted on the cells in different ways including hydraulic loading of cells encapsulated in a gel supported by a plastic ring; by placing cells in culture or encapsulated in gels in a sealed chamber and applying pressure by influx of 5% CO₂ or through the application of balanced air at various pressures and delivery cycles, especially at pressures up to 7 psi (0.048 MPa) at varying frequencies (e.g. 3 seconds on and off or 5 seconds on and off) for different time periods (2-4 hours).

The application of load according to the regimens disclosed above and in the Examples represent a further important aspect of the invention. Therefore according to a sixth aspect of the invention there is provided a method for causing mesenchymal stromal stem cells to differentiate towards IVD cells comprising exposing cultured mesenchymal stromal stem cells to increasing pressures of up to 7 psi (0.048 MPa).

(D) Co-culture of MSSCs with Nucleus Pulposus (NP)cells/IVD cells—The inventors have established that normal NP cells and mesenchymal stromal stem cells (MSSCs) may be cultured together (with or without contact), with the result that the MSSCs differentiate towards IVD cells. Alternatively the MSSCs may be cultured in conditioned media (i.e. media in which NP cells have previously been grown).

The technique disclosed above and in the Examples (see 1.1.3.5) represent a further important aspect of the invention. Therefore according to a seventh aspect of the invention there is provided a method for causing mesenchymal stromal stem cells to differentiate towards IVD cells comprising co-culturing NP cells and mesenchymal stromal stem cells (MSSCs) together but without contact between the two cell types.

According to an eighth aspect of the invention there is provided a method for causing mesenchymal stromal stem cells to differentiate towards IVD cells comprising culturing mesenchymal stromal stem cells in media that has previously been exposed to NP cells.

A preferred co-culturing technique for differentiating cells to, or towards, IVD cells is illustrated in Example 6.

It is preferred that conditioned media used for activation/differentiation of MSSCs is prepared from early passage (e.g. P1 and P2) of normal NP cells and then concentrated using a centrifugal filter system. The concentrated media is then used in culture at concentrations ranging from 1-30% in standard media to induce differentiation.

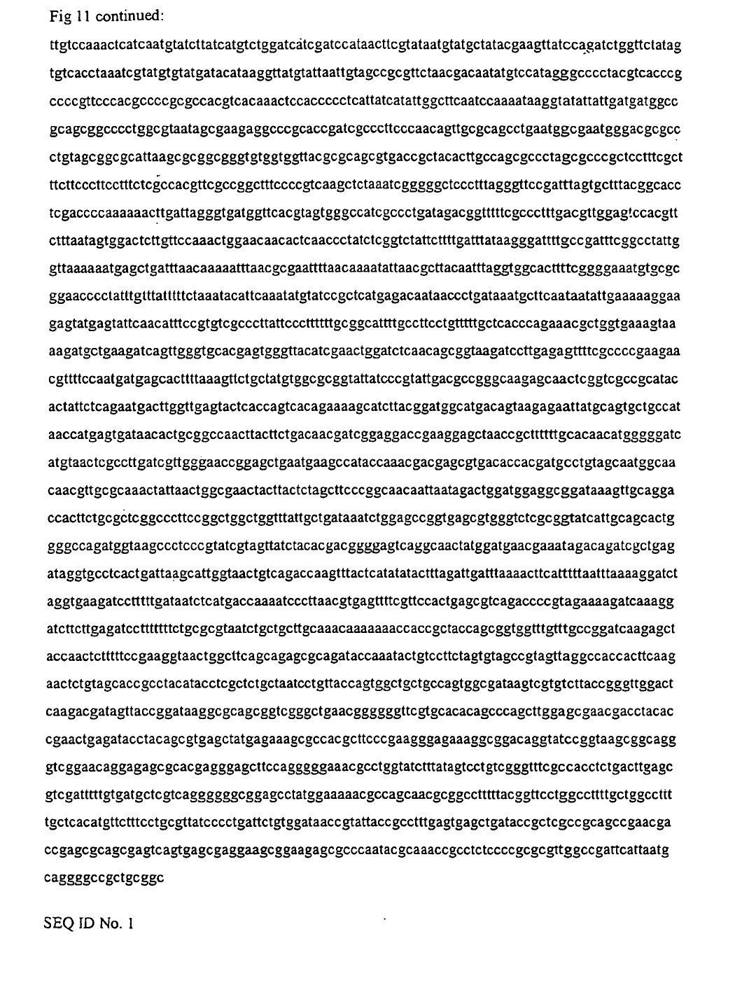

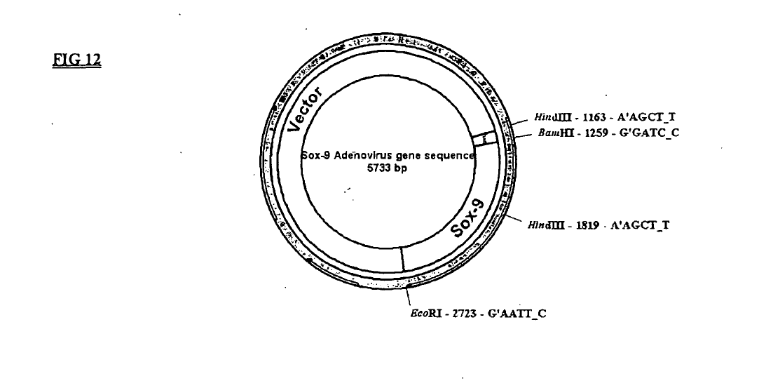

(E) Altering the gene expression profile of the cell using gene therapy—This involves inserting a gene that regulates development of an NP phenotype (especially TGFβ, Sox-6, Sox-9) into an MSSC using a viral or non-viral vector that may either integrate or not integrate into the DNA of the MSSC. It is preferred that the exogenous gene is Sox-9. It is preferred that a Sox-9 gene with the sequence contained within SEQ ID No 1 (see FIG. 11), or the gen sequnec identified obn gene bank as accession number z46629, is used to infect the MSSCs. It is most preferred that the SOX-9 gene is inserted in a viral expression vector (see below) as illustrated in FIG. 12. According to a further aspect of the invention there is provided a method for causing mesenchymal stromal stem cells to differentiate towards IVD cells comprising inserting into the MSSC a gene, and particularly the SOX-9 gene, in an expression vector that regulates the development of an NP phenotype.

(F) Oxygen tension—The inventors have also found that altering the oxygen tension of cultured MSSC cells, and particularly making them hypoxic, may promote differentiation towards IVD cells.

For instance, MSSC may be cultured in a monolayer in media (e.g. by conventional techniques; as described in (A) above; or the Examples) in 0.1-20% O₂ for up to 4 weeks. Differentiation may be promoted at less than 10% O₂; preferably at less than 5% O₂ and more preferably at about 1% O₂—Cells should be cultured at these oxygen tensions for at least a day, preferably at least 3 days and more preferably for about 1 week (or more). Data illustrating the usefulness of this differentiation procedure is given in Example 3.

The oxygen tension technique described above represents a further important aspect of the invention. Therefore according to an eighth aspect of the invention there is provided a method for causing mesenchymal stromal stem cells to differentiate towards IVD cells comprising culturing mesenchymal stromal stem cells (MSSCs) in an atmosphere comprise 5% or less Oxygen.

It will be appreciated that differentiation steps (A)-(F) may be used singularly or in combination to induce MSSCs to differentiate into cells with an IVD cell phenotype.

According to a preferred general differentiation procedure, the MSSCs are conditioned to adopt an NP phenotype by placing them in a simple gel (such as alginate) and loading them (as described above) whilst being cultured in a differentiation medium. The medium may: (i) contain a growth factor (e.g. TGFβ or CDMP); (ii) be a conditioned-media as described above); or comprise IVD cells (i.e. a co-culture medium).

A preferred procedure comprises resuspending cells in 1.2% medium viscosity alginate in 0.15M NaCl at a density of about 5×10⁶ cells/ml and then polymerising the alginate to form a layer of encapsulated cells. The encapsulated cells may be grown in a medium containing TGFβ or in a conditioned medium (as described above), for between 2 days and 6 weeks during which a load is exerted using a cyclical compressive load of 2 to 100 psi at between 0.1 and 2 (typically 0.5) Hz.

According to another preferred differentiation procedure, MSSC may be encapsulated in alginate; incubated in media as described above; and exposed to a dynamic cyclical compressive load (0-4 mPa) at a rate of between 0.5 and 3 Hz for between 1 and 35 days. More preferably the load may be applied as 0.8-1.7 mPa at 1 Hz for 4 hours repeated every 48 hours for 7 days. Data illustrating the usefulness of this differentiation procedure is given in Example 4.

Cells differentiated according to the first aspect of the invention may be used for a number of therapeutic uses. For instance, the cells may be administered to a diseased intervertebral disc of a subject in need of such treatment or by seeding onto or into a biomaterial in vitro to be implanted or injected into the patient's intervertebral disc. The biomaterial may be a natural compound or material (e.g. collagen) or may be a synthetic material.

According to a preferred embodiment of the invention undifferentiated pluripotent mesenchymal stromal stem cells (MSSCs according to the invention) may be differentiated toward IVD cells. Such cells may be used to repopulate the IVD with viable cells or may also be used to seed biomaterial scaffolds and gels for the management of DIVD according to the invention. These three therapeutic uses may be combined as the clinical need dictates.

Briefly autologous undifferentiated pluripotent mesenchymal stromal cells may be harvested from the patient's own marrow, peripheral blood and/or adipose tissue and then grown to confluence in vitro. The MSSCs are then transferred to a medium in which they will be differentiated into nucleus pulposus (NP) cells according to the preferred procedures described above. After this the now differentiated and conditioned MSSC-derived IVD cells will be used to:
(a) Repopulate the IVD by direct injection into the diseased IVD with the purpose of their producing fresh matrix; or
(b) Seed biomaterial scaffolds and gels in vitro to produce tissue engineered implants suitable for restoring IVD matrix and/or function.

The inventors have also realised that the IVD is avascular. This limits access of oral or systemically delivered pharmaceutical agents to native IVD cells. Furthermore repeated injection of such agents directly into the IVD is also problematic because of the proximity to important organs (eg. aorta, spinal cord) and the risk of introducing infection. Accordingly the inventors have recognized that there is need for providing improved means for delivering therapeutic agents to the IVD. This lead to a further aspect of the invention, described in more detail below, whereby cells according to the first aspect of the invention may be adapted for the delivery of therapeutically active agents.

The inventors work has shown that biological agents (eg. IL-1Ra, TGF, TIMPs) could be used therapeutically raising the possibility of a gene therapy approach to managing DIVD. Within the current climate the use of in vivo gene therapy for treating spinal conditions raises a number of problems. Using the in vivo approach would entail injecting an expression vector (usually a viral vector) into the IVD at an unknown concentration (in terms of vectors per cell). The addition of viral vectors at high MOIs has cytotoxic effects, and as cell number in the degenerate IVD is unknown and cannot be determined easily, the addition of toxic concentrations of viral vector would be a clear possibility. In addition the mis-injection of viral vector into: (a) a blood vessel may result in a systemic immune response, which could be fatal; and (b) a recent study by Wallach et al., (2003: 84 International Society for the Study of the Lumbar Spine, Conference Abstract), demonstrated the strong possibility of inducing paralysis following mis-injection of a TGF D adenoviral vector at high dose into areas surrounding the IVD (Wallach et al. 2003 supra)

The use of an ex vivo gene therapy approach (i.e. in vitro transformation of cells with a therapeutic gene and then introducing the cells into a subject) has been contemplated for the treatment of degenerative back conditions. This approach has a number of advantages over the use of in vivo gene transfer. One of the clearest advantages of such an approach would be the ability to perform extensive safety controls before the insertion of cells into the IVD. In addition higher levels of transgene could potentially be produced, as cells could be selected prior to injection into the IVD for production of high levels of therapeutic protein. One attempt at an ex vivo gene therapy approach to the treatment of spinal conditions is disclosed in DE4219626.

However to date none of these in vivo or ex vivo gene therapy approaches have been successful enough to be used in the clinic. The inventors have found that IVD cells produced from MSSC may be more successful in this capacity and according to a ninth aspect of the present invention there is provided an isolated mesenchymal stromal stem cell (MSSC) that has been:
(a) differentiated in vitro towards an IVD cell phenotype; and
(b) transfected with an exogenous gene which codes for a protein that reduces degeneration of an intervertebral disc.

MSSC/IVD cells according to the first aspect of the invention may be genetically transformed with the exogenous gene before or after the process of full differentiation has been completed.

The exogenous gene must be cable of being expressed from the cells (preferably in vitro as well as when administered to a subject) to produce a protein that directly or indirectly has activity for reducing degeneration of an intervertebral disc. By “directly” we mean that the product of gene expression per se has the required activity. By “indirectly” we mean that the product of gene expression undergoes or mediates (e.g. as an enzyme) at least one further reaction to provide an agent effective for reducing degeneration of an intervertebral disc.

The exogenous gene may be selected from a number of genes that have known activity for reducing inflammation. These include: cytokines (especially molecules of the TGFβ superfamily); inhibitors of cytokines (especially the specific interleukin-1 inhibitors, interleukin-1 receptor antagonist [IL-1ra] and the soluble type II interleukin-1 receptor [IL-1RII]) and inhibitors of degradative enzymes, (especially TIMPs 1, 2 and 3 and other inhibitors of matrix metalloproteinases and ADAMTs)

A preferred exogenous gene codes for the Interleukin 1 Receptor Antagonist (IL-1RA). IL-1RA is a natural inhibitor of IL-1. The inventors have shown that IL-1RA is produced by normal intervertebral disc cells together with IL-1 but in an excess over the cytokine that would inhibit IL-1 activity. However they have demonstrated in diseased IVD that IL-1 but not IL-1RA production is up-regulated. The implication of this finding is that there is a relative shortfall in IL-1RA production in spinal conditions according to the invention. Therefore the exogenous gene preferably codes IL-1RA.

The exogenous gene may be contained within a suitable vector to form a recombinant vector. The vector may for example be a plasmid, cosmid or phage. Such recombinant vectors are highly useful for transforming cells with the exogenous gene.

Recombinant vectors may also include other functional elements. For instance, recombinant vectors may be designed such that the vector will autonomously replicate in the nucleus of the cell. In this case, elements that induce DNA replication may be required in the recombinant vector. Alternatively the recombinant vector may be designed such that the vector and recombinant DNA molecule integrates into the genome of a cell. In this case DNA sequences that favour targeted integration (e.g. by homologous recombination) are desirable. Recombinant vectors may also have DNA coding for genes that may be used as selectable markers in the cloning process.

The recombinant vector may also further comprise a promoter or regulator to control expression of the gene as required.

The exogenous gene may be inserted into a retroviral vector. Such vectors may advantageously fully integrate into the host genome. This results in long-term gene expression, with integrated genes passed onto daughter cells.

Recently however a gene therapy patient, undergoing treatment for severe combined immune deficiency (SCID) using a retroviral vector, was cured of SCID but developed a leukaemia-like condition thought to be caused by ‘insertional mutagenesis’ of the retrovirus. In addition retroviruses have also been shown to cause leukaemia within mice. This suggests that it may be detrimental to use retroviral vectors. It is therefore preferred that the exogenous gene is inserted in an adenoviral vector. The use of adenoviral vectors avoids the risk of insertional mutagenesis as the vector remains episodic and is not integrated into the genome. In addition the Ad vector has good transduction ability to quiescent, non-dividing, highly differentiated cells.

The inventors believe that adenoviral vectors are useful in an ex vivo approach to gene transfer in the IVD. In most cases of gene transfer the inability to integrate into the genome by adenoviruses is considered to result in the short life of the transgene, as it is lost during cellular division. However, within the IVD where cellular turnover rates are very low this will not cause the same problems, and long term gene expression is possible.

According to a tenth aspect of the invention there is provided a recombinant vector suitable for genetically transforming mesenchymal stromal stem cells, or IVD cells produced from them according to the ninth aspect of the invention, comprising an adenoviral expression vector containing a gene encoding a protein that reduces DIVD.

Preferred recombinant vectors according to the tenth aspect of the invention are described in Example 2.

It will be appreciated that the exogenous gene may be delivered to the MSSC without it being incorporated in a vector. For instance, the exogenous gene may be incorporated within a liposome or virus particle. Alternatively the “naked” DNA molecule may be inserted into the MSSCs by a suitable means e.g. direct endocytotic uptake.

The exogenous gene (contained within a vector or otherwise) may be transferred to the MSSCs (before or after differentiation towards IVD cells) by transfection, infection, microinjection, cell fusion, protoplast fusion or ballistic bombardment. For example, transfer may be by ballistic transfection with coated gold particles, liposomes containing the exogenous gene, and means of providing direct DNA uptake (e.g. endocytosis).

It is preferred that the cells are infected with the exogenous gene during the differentiation process.

When the cells are encapsulated in gel it is preferred that infection occurs during gel encapsulation. Cells to be infected in gel are preferably infected via the addition of viral suspension to gel cell suspension before polymerisation. A most preferred infection protocol is given in Example 1.

It will be appreciated that cells according to the ninth aspect of the invention may be used for the same purposes as those described above for those according to the first aspect of the invention. Furthermore the cells according to the ninth aspect of the invention have the advantage that they have been engineered to contain a therapeutically effective agent that may have efficacy against DIVD.

The invention will be illustrated further by Example and with reference to the following drawings, in which:

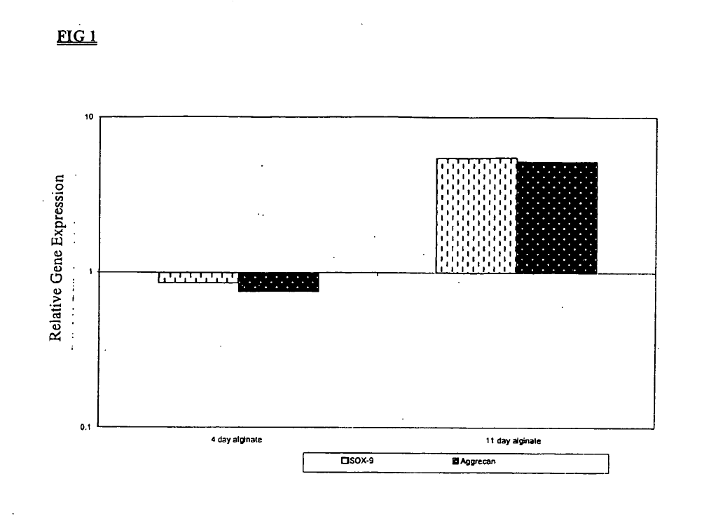

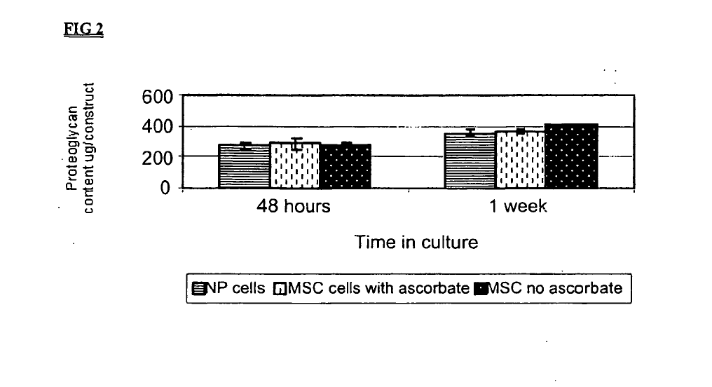

FIG. 1 illustrates the Level of gene expression for Sox 9 and aggrecan in MSSCs cultured in IVD cell inducing media as described in Example 1;

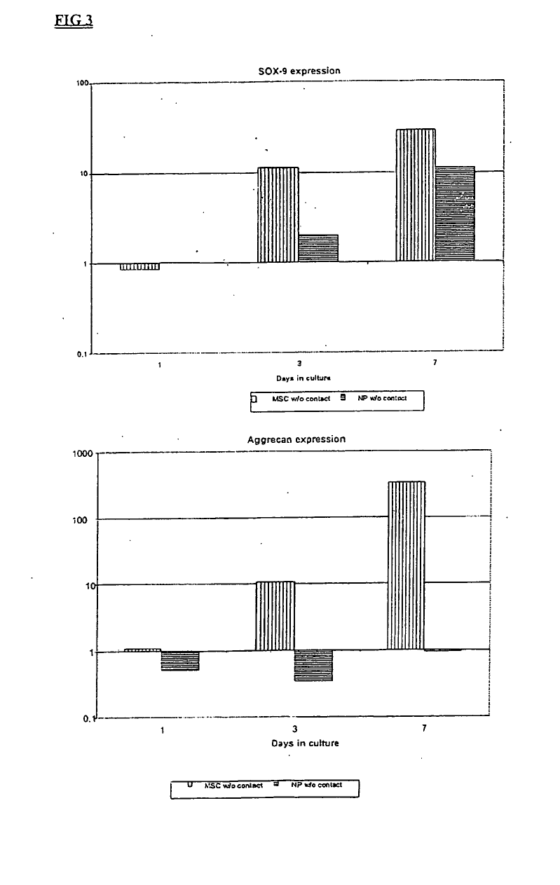

FIG. 2 illustrates proteoglycan production in gel constructs loaded at 5 psi 3 sec on 3 sec off for 2.5 hours as described in Example 1;

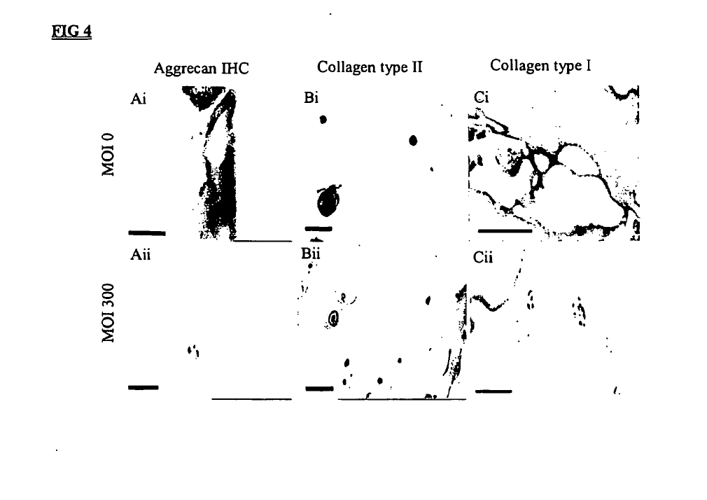

FIG. 3 illustrates results from Real-time PCR showing changes in SOX-9 mRNA expression (A) and aggrecan mRNA expression (B) in monolayer MSSC and NP cells over time (samples normalised to GAPDH and cells in single population culture) as described in Example 1;

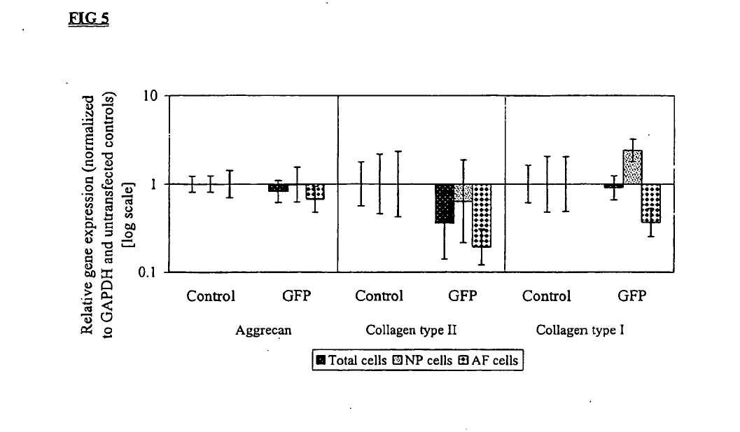

FIG. 4: Immunohistochemical staining for aggrecan (A), collagen type II (B), or collagen type I (C) within alginate bead cultures infected with Ad-LacZ (ii) and uninfected cells (i) (Bars=380 nm) as described in Example 2;

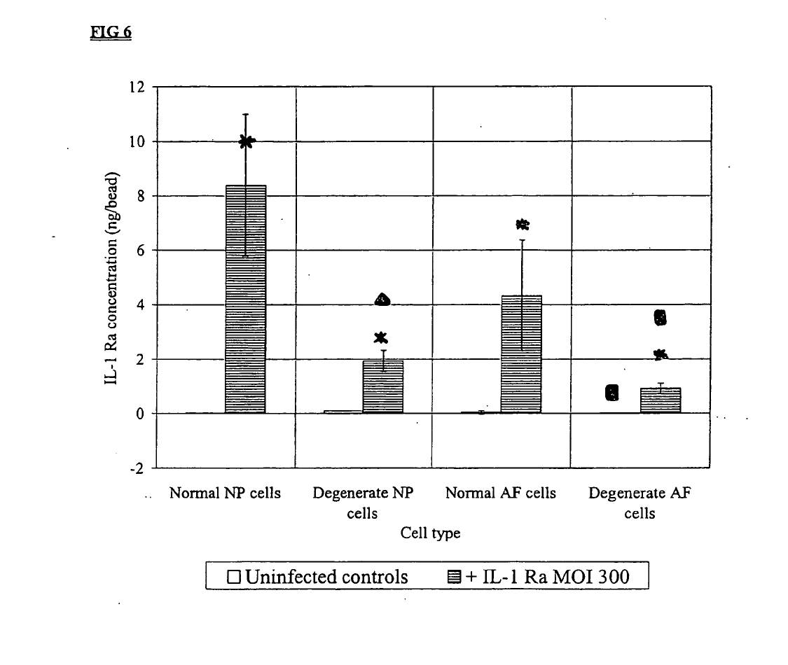

FIG. 5: Gene expression of the matrix proteins aggrecan, collagen type II and collagen type I following infection of cells with Ad-GFP within alginate beads 5 weeks post infection as described in Example 2;

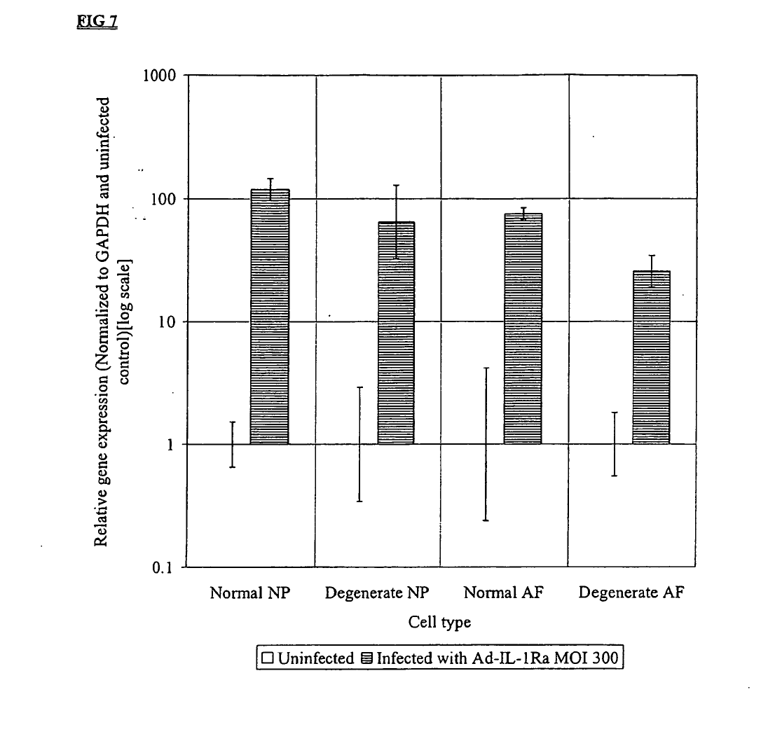

FIG. 6: IL-1 Ra protein production 2 days post infection within cells infected with IL-1R^{a} MOI 300 cultured in. Error bars=standard error of the mean. Statistical analysis p<0.1 for Normal AF cells, p<0.05=all other cells, *=Probability values for difference between uninfected and infected cells. ▪=Probability values for difference between NP and AF cells. ▴=Probability values for difference between normal and degenerate cells as described in Example 2;

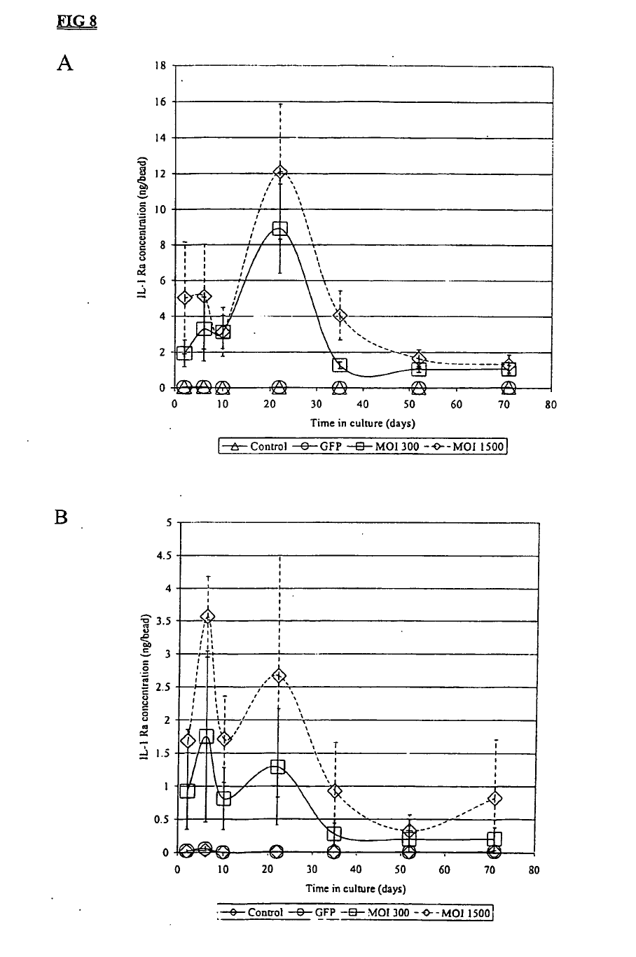

FIG. 7: Gene expression of IL-1 Ra following infection within alginate culture and culture within alginate beads for 5 weeks; Error bars=Standard error of the mean; and statistical significance as described in Example 2;

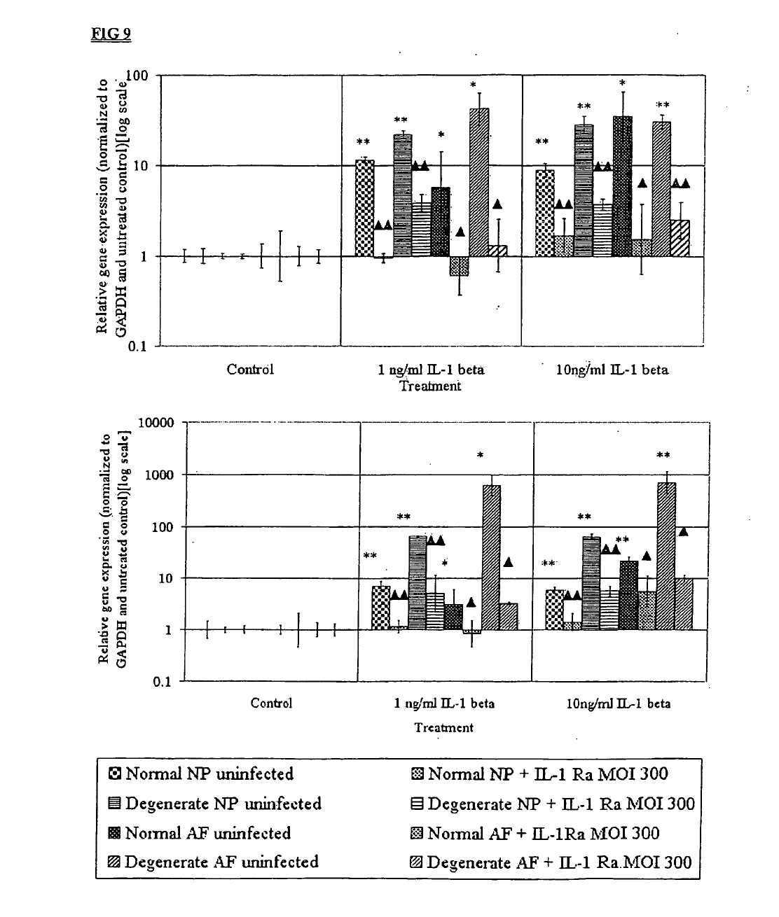

FIG. 8: IL-1Ra production in degenerate NP (A) and AF (B) cells infected in alginate culture for 72 days as described in Example 2;

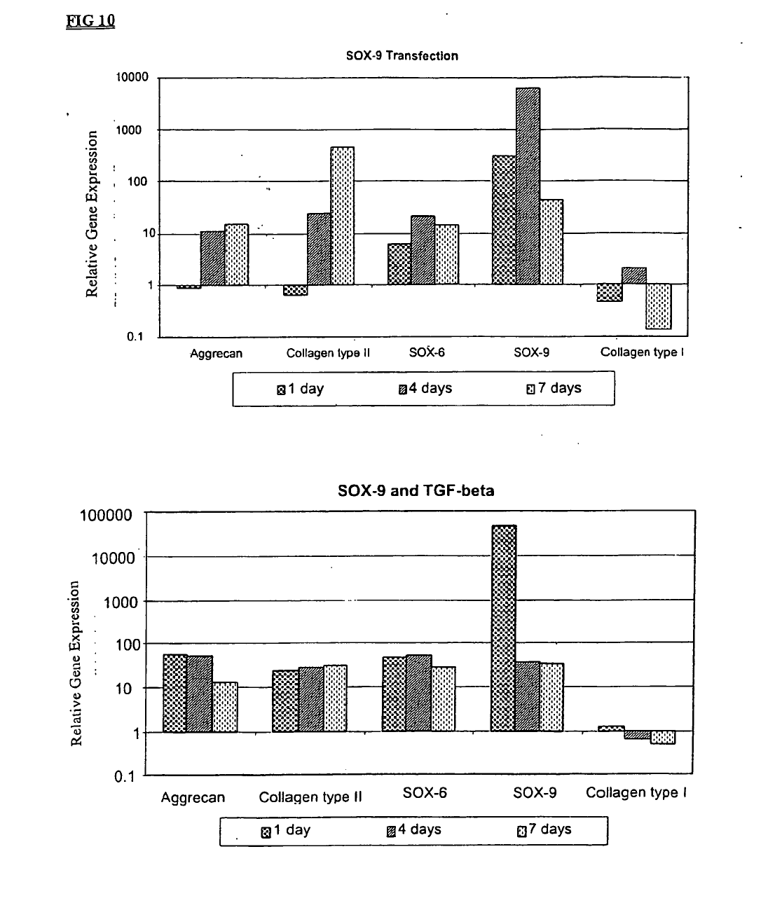

FIG. 9: Gene expression of MMP 3 (A) and MMP 13 (B) in IVD cells (uninfected and infected with Ad-IL-1Ra (MOI 300)) treated with IL-1β and cultured in monolayer; Error bars=standard error of the mean; Statistical analysis p<0.1=* or ▴, p<0.05=** or ▴▴; Asterisks=probability values for difference between untreated and treated cells; Triangles=probability values for difference between uninfected and infected cells as described in Example 2;

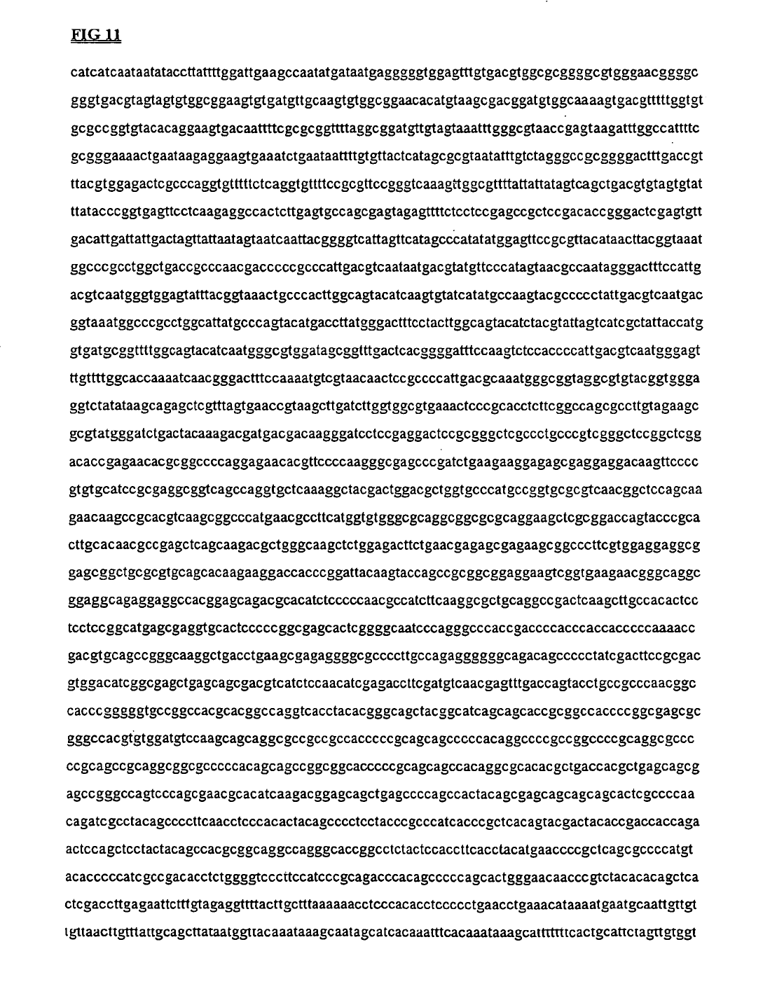

FIG. 10: Real-time PCR data showing relative gene expression of matrix component molecules after SOX-9 tranfection (A) and SOX-9 transfection with TGF-β1 stimulation (B) of monolayer MSSCs. Samples are normalised to GAPDH and GFP-transfected, unstimulated controls as described in Example 2;

FIG. 11: represents a sequence of a vector comprising Sox 9 gene as described in Example 2;

FIG. 12: represents a vector map of a preferred expression vector containing Sox 9

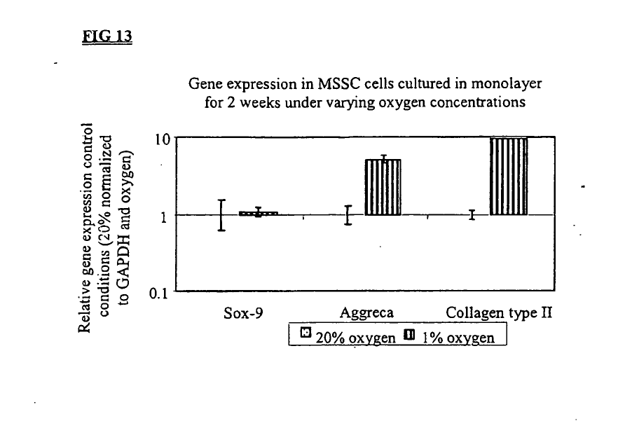

FIG. 13: is a bar chart illustrating gene expression of Sox-9, aggrecan and Collagen type II in MSSC cells cultured in monolayer in 1% and 20% oxygen for 2 weeks according to Example 3;

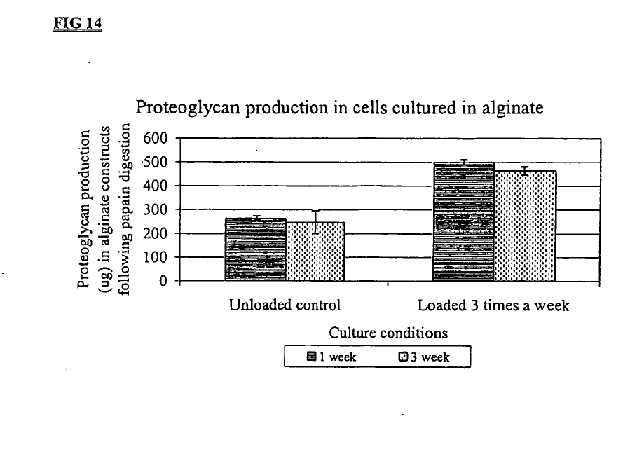

FIG. 14 is a bar chart illustrating proteoglycan production (a marker of differentiation towards NP cells) using a preferred differentiation technique described in Example 4; and

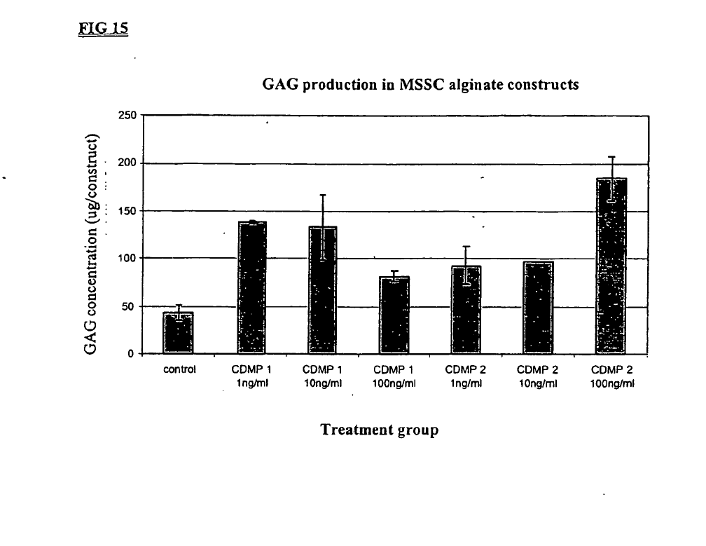

FIG. 15 is a bar chart illustrating that CDMP treatment of MSSC cells increased proteoglycan production as described in Example 5.

### EXAMPLE 1

### Mesenchymal Stromal Stem Cell Harvesting, Cell Culture and IVD Cell Differentiation

1.1 Methods

1.1.1 Mesenchymal Stromal Stem Cell Harvesting

Mesenchymal stromal stem cells were harvested from iliac crest bone marrow aspirates. 5-10 ml of marrow are placed into 4.45 ml HBSS, 0.05 ml of PSA and 0.5 ml of heparin (100 units) at the time of harvest. The sample is centrifuged at 500 g for 10 minutes. The supernatant is removed and the pellet resuspended in 5 ml of αMEM. The cell suspension is then transferred to a 15 ml centrifuge tube and 5 ml of histopaque 1077 added to the tube below the medium. The tube is centrifuged at 500 g for 30 minutes to isolate the stromal cells. Following centrifugation the grey interface layer containing the mononuclear cells is removed by gentle aspiration, passed through a cell strainer and resuspended in standard media (αMEM, 10% FCS, 1% glutamine, 1% ascorbate and 1% penicillin, streptomyocin and amphotericin in T75 culture flask. Cells are incubated at standard culture conditions (37° C. in 5% CO₂). Cells are left to settle for 1 week without any change of media, and then the media is removed, cells washed in PBS and fresh media added. Media thereafter is changed every 2-3 days, and cells expanded through 2-3 passages by standard methods.

1.1.2 Cell Culture of IVD Cells

The IVD cells are cultured in DMEM+F12 media supplemented with 10% heat inactivated fetal calf serum (FCS) (Gibco), 100 U/ml Penicillin (Sigma), 100 μg/ml Streptomycin (Sigma), 250 ng/ml amphotericin, 2 mM glutamine (Sigma) and 50 μg/ml ascorbic acid (Sigma) in T75 flasks (Appleton woods) and cultures maintained at 37° C. in a humidified atmosphere containing 5% CO₂. Culture media is changed every other day.

1.1.3 Production of IVD Cells from Mesenchymal Stem Cells

A number of differentiating techniques were used singularly or in combination:

1.1.3.1 IVD cell induction medium—DMEM/HAMs F12 1:1 supplemented with 1.349 g glucose, 50 μg/ml L-ascorbic acid, 100 units/ml penicillin, 100 μg/ml ml streptomyocin, 250 μg/ml 250 ng amphotericin, 10 ng/ml TGF-β3, 1×10⁻⁷ M dexamethosone, 100 μg/ml sodium pyruvate, 40 μg/ml proline and 1% ITS+1 premix. (ITS mix from Sigma with a premix comprising 1 mg/ml insulin, 0.55 mg/ml transferin, 0.5 μg/ml sodium selinite; 50 mg/ml BSA and 470 μg/ml linoleic acid)

1.1.3.2 Alginate encapsulation—Monolayer stromal cells are trypsinized and the resulting cell suspension centrifuged at 500 g. The cell pellet is then resuspended in 10 ml of stromal media, and cells counted using a coulter counter. Following another centrifugation at 500 g the cells are then resuspended in 1.2% medium viscosity alginate in 0.15M NACl at a density of 1×10⁶ cell/ml. The resulting suspension is then expressed through a 22 gauge needle using a 5 ml syringe in to 12 well plates containing 102 nM CaCl₂ to form polymerized microspheric beads. After 10 minutes the polymerized alginate is washed in normal saline, followed by a wash in standard medium.

1.1.3.3 Induction of IVD cellphenotype—Cells encapsulated in alginate are then placed in the IVD cell induction media and cultured under standard conditions for up to 12 days.

1.1.3.4 Application of Load—Cells are loaded within 24 well plates in alginate constructs. The plate is placed into a sealed chamber and pressure applied by influx of 5% CO₂, balanced air at pressures up to 7 psi (0.048 MPa) at varying frequencies (3 seconds on and off, and 5 seconds on and off) for different time periods (2-4 hours).

1.1.3.5 Co culture of MSSCs with Nucleus Pulposus (NP)cells—Normal NP cells (P3) and MSSCs (P3) are grown in monolayer to confluence and then trypsinised and counted. Cells are then cultured together but without contact, in the ratio of 60,000 NP cells and 30,000 MSSCs. For the without contact wells, NP cells are pipetted into the bottom of the wells and then a tissue culture insert is placed in the well and MSSCs pipetted onto the insert. 2 mls DMEM-F12+FCS are then added to each well and cells Incubated under standard conditions.

1.2 Results

The Inventors conducted experiments that confirmed that cells used in the art were unsuitable for the treatment of degenerative IVDs.

Key to the strategy of repair and tissue engineering of the IVD is the generation of suitable cells for injecting into and regenerating the NP. One approach has been to harvest cells from the patient's IVD. Although many workers use these cells, the inventors surprisingly found that this approach is flawed because the cells have a senescent phenotype, cannot withstand the loads of the IVD, and when infected with IL-1RA adenovirus and injected into explant IVD cannot increase IL-1RA in the tissue.

1.2.1 Evidence of Cellular Senescence

The inventors investigated cell replication potential (which falls with senescence) and expression of the cyclin-dependent kinase inhibitor P16^{INK4A} protein (upregulated during cellular senescence), in tissue and cells of normal and degenerate NP. This showed age-related: decrease in replication potential; and increase in cellular expression of P16^{INK4A}; in the normal NP. NP cells of degenerate IVD, however, even from young patients, exhibited a decrease in replicative potential and increased expression of P16^{INK4A} equivalent to those of normal individuals as much as 40 years older. In addition they have shown by real time RT PCR that there is a direct relationship between expression of senescent biomarkers and the genes for: the matrix degrading enzymes MMPs 3 & 13; the aggrecanase, ADAMTs 5; and type I collagen; and an inverse relationship with expression of type II collagen and aggrecan. Such data suggest that degenerate NP cells show a senescent phenotype with an altered cellular metabolism that may prevent their subsequent use in repairing the IVD.

1.2.2 Effect of Load

Preliminary data shows that degenerate NP cells appear to be hypersensitive to loading at pressures as low as 1 psi (0.0069 Mpa—the minimum load on the intervertebral disc is 0.3 Mpa). Our experiments also show that cellular responses are dependent on cell type and the frequency and amount of load applied. For instance significant differences were observed in gene expression of degenerate NP cells loaded at 5 psi (0.0345 MPa) and at 7 psi (0.0483 MPa)

1.2.3 IL-1RA Adenoviral Infected IVD Cells

The ability of cells infected with Ad-IL-1Ra to contribute to increased total production of IL-1Ra in the tissue was also studied. Cells derived from degenerate and non-degenerate IVD were infected with Ad-IL-1Ra, labeled with CFDA-SE and injected into explants of degenerate human IVD. CFDA-SE labelled cells were identified in all tissue explants. When normal IVD cells infected with Ad-IL-1Ra were injected into the tissue explants a significant increase in IL-1Ra immuno-positive cells was observed. However, when Ad-IL-1Ra infected degenerate cells were injected into the tissue explants no increase in the proportion of IL-1Ra immunopositive cells was observed. This demonstrates that the use of degenerate IVD cells may be inappropriate for the transfer of IL-1Ra to the degenerate IVD.

1.2.4 Production of IVD Cells from MSSC

Mesenchymal cells cultured in alginate and treated with IVD cell inducing media show increased gene expression of Sox 9 and aggrecan after 11 days (FIG. 1)

1.2.5 Effect of Load in Inducing an IVD Cell Phenotype

Culture of MSSC cells in alginate cultures under compressive load resulted in equivalent proteoglycan production to NP cells cultured under the same conditions, with an increase in proteoglycan content with time in culture. (FIG. 2)

1.2.6 Co-Culture of MSSC with NP Cells

Co-culturing MSSC with normal NP cells led to differentiation of the MSSC into NP cells. In addition real-time PCR showed that both the NP cells and MSSCs increased expression of SOX-9 gene (FIG. 3). Cells were maintained without contact, suggesting factors released by NP cells will cause MSSC to differentiate into NP cells.

### EXAMPLE 2

### Gene Transfer

This example illustrates the work conducted by the inventors to insert an exogenous gene (IL-1RA) into dedifferentiated NP cells. NP cells when passaged 2-3 times in culture tend to spontaneously dedifferentiate in culture towards a MSSC phenotype. Accordingly, such dedifferentiated cells represent a good model of MSSCs according to the invention.

2.1 Choice of Viral vectors—The retroviral vector's capability to fully integrate into the host genome results in long term gene expression, with integrated genes passed onto daughter cells. However mutagenesis is a possibility if viral insertion was to occur in a non-favourable site. Recently within a gene therapy patient in France, who was undergoing treatment for severe combined immune deficiency (SCID) using a retroviral vector, was cured of SCID but developed a leukaemia like condition thought to be caused by ‘insertional mutagenesis’ of the retrovirus. In addition retroviruses have also been shown to cause leukaemia within mice. These studies appear to be detrimental to the use of retroviral gene transfer. The use of adenoviral vectors avoids the risk of insertional mutagenesis as the vector remains episodic and is not integrated into the genome. In addition the Ad vector has good transduction ability to quiescent, non-dividing, highly differentiated cells. Adenoviral vectors may be useful in an ex vivo approach to gene transfer in the IVD. In most cases of gene transfer the inability to integrate into the genome by adenoviruses is considered to result in the short life of the transgene, as it is lost during cellular division. However, within the IVD where cellular turnover rates are low this may not cause the same problems, and long term gene expression may be possible. In addition, loss of adenoviral gene transfer is often caused by immune reactions to viral proteins, or to the foreign proteins encoded by the transgenes. However this may not cause a problem within the IVD, as the IVD is considered an immuno-privileged site.

Viral Vector-Ad-IL-1Ra vector was a kind gift from Professor Christopher H Evans (Harvard University, Boston). Briefly IL-1Ra cDNA was cloned from a human monocyte cDNA library as detailed by Bandara et al., (1993 Proc, Natl. Cad. Sci USA 90: 10764-10768). First generation, E1, E3-deleted serotype 5 recombinant adenoviral vector containing IL-1Ra was then constructed using Cre-lox recombination by the system of Hardy et al., (1997 J Virol 71, 1842-1849). All three adenovirus constructs were grown within 293 cells, and following lysis, the viral suspension was purified using CsCl density gradient purification (Bet, Prevec et al. J Virol 67, 911-5921).

2.2 Infection Protocol—Preferred timing of infection is during alginate encapsulation. Cells to be infected in alginate are infected via the addition of viral suspension (correct volume calculated as for monolayer) to alginate cell suspension before polymerisation. In brief following expansion in monolayer culture cells are trypsinized and cell suspensions spun at 300 g for 5 minutes to generate a cell pellet. The cell pellet is then resuspended in 10 ml of DMEM+F12 and cell number counted using a coulter counter ZM (Coulter electronics). Following further pelleting, cells are resuspended in 1.2% low viscosity sodium alginate in 0.15M NaCl at a density of 1×10⁶ cells/ml and appropriate amount of viral suspension added. Following mixing the cell/viral suspension is passed through a 23 gauge needle into a 12 well plate containing 200 mM CaCl₂ where each drop was instantly polymerised forming semisolid microspheric beads. Beads are incubated for 10 minutes at 37° C. to allow further polymerisation, followed by two washes in 0.15M NaCl and two washes in serum free media. Two millilitres of IVD cell induction media is added to each well and cultures maintained at 37° C. in a humidified atmosphere containing 5% CO₂.

2.3 Effect of IL-1RA Gene Transfer on Cells

2.3.1 Phenotypic stability of virally infected cells No difference was seen between Ad-LacZ infected cells and non-infected cells in the staining pattern of any of the matrix proteins investigated (aggrecan (p=0.499), collagen II (p=0.5) and collagen type I (p=0.498)). With high proportions of cells positive for type II collagen and aggrecan and low proportions of positive cells for collagen type I even after 4 weeks in culture (FIG. 4). Similar results were seen in cells infected with Ad-GFP with no significant difference within any cell type in any of the matrix proteins investigated, compared to non-infected cells (FIG. 5) (probability values for total cells: Aggrecan p=0.786, Collagen type II p=0.954, Collagen type I p=0.3588).

2.3.2 IL-1Ra Production Following Gene Transfer

Infection of cells in alginate resulted in a significant increase in IL-1Ra protein 48 hours post infection in all cell types investigated (normal NP p<0.05, degenerate NP p<0.05, normal AF p<0.1 and degenerate AF p<0.05) compared to IL-1Ra production in uninfected cells (FIG. 6).

Uninfected degenerate NP cells showed significantly higher levels of IL-1Ra than uninfected degenerate AF cells (p<0.05). In addition Ad-IL-1Ra infected degenerate NP cells also showed higher IL-1Ra protein than Ad-IL-1Ra infected degenerate AF cells (p<0.05) (FIG. 6). Normal IVD cells did not show any significant difference in IL-1Ra production between NP and AF cells. Uninfected normal cells did not show any significant difference in IL-1Ra production to uninfected degenerate IVD cells. However, Ad-IL-1Ra infected normal cells showed higher IL-1Ra production than infected degenerate cells (NP cells (p<0.05), AF cells (p>0.1)) (FIG. 6). Following culture for a further 5 weeks in alginate cells infected with Ad-IL-1Ra showed elevated IL-1Ra gene expression in all cell types (normal NP p<0.05, degenerate NP p<0.1, normal AF p<0.05, degenerate AF p<0.05) compared to uninfected cells (FIG. 7).

2.3.4 Length of Transgene Expression in Alginate Culture

Degenerate NP cells infected with Ad-IL-1Ra (MOIs 300 and 1500) in alginate produced significantly higher levels of IL-1Ra protein than uninfected control cells 2 days post infection (MOI 300 p<0.05, MOI 1500 p<0.05) and at all subsequent time points (all p values <0.05). Levels were significantly higher in cells infected at an MOI of 1500 than those infected at an MOI of 300 (p<0.05) (FIG. 8A). Cells infected with Ad-GFP did not show any significant difference in IL-1Ra protein production over the time course investigated (all p values >0.1) (FIG. 8A).

Degenerate AF cells infected with Ad-IL-1Ra (MOI 300 and 1500) in alginate also showed significantly higher levels of IL-1Ra protein compared to uninfected control cells 2 days post infection (MOI 300 p<0.05, MOI 1500 p<0.05). In addition significantly higher levels of IL-1Ra protein were seen in cells infected with Ad-IL-1Ra at an MOI of 1500 than those infected with an MOI of 300 (p<0.05) (FIG. 8B). No difference in levels of IL-1Ra protein were seen in Ad-GFP infected cells compared to uninfected controls (all p values >0.1). At all time points studied, degenerate AF cells infected with Ad-IL-1Ra (MOI 300 & 1500) showed no significant decrease in levels of IL-1Ra from those produced following 2 days post infection (all p values >0.1). Normal NP and AF cells infected with Ad-IL-1Ra (MOI 300) showed significantly higher levels of IL-1Ra protein than uninfected control cells (p<0.05) 2 days post infection. After 5 weeks in alginate culture no significant change in levels of protein were seen. Levels of IL-1Ra protein in infected cells remained significantly higher than control cells even following 5 weeks in alginate culture (p<0.05).

2.3.5 Inhibition of IL-1 with IL-1Ra

Treatment of IVD cells in monolayer culture for 48 hours with IL-1β resulted in a significant increase in the gene expression of MMP 3 and MMP 13 (FIG. 9). In all cell types the response to IL-1β was inhibited by infection of cells with Ad-IL-1Ra (MOI 300) with decreased MMP 3 and MMP 13 gene expression (FIG. 9).

2.4 Transfection of Dedifferentiated NP Cells with Sox-9 Adenovirus

Transfection of monolayers of dedifferentiated NP cells with adenoviral SOX-9 led to their developing an NP phenotype in monolayer that was improved by addition of TGF-β1 (FIG. 10). The results show TGF-β-stimulated, SOX-9 transfected, monolayer MSSCs, produce a large population of dividing cells that have an NP-like phenotype that are readily accessible for use in tissue engineering applications

The sequence of the Sox-9 vector used in this Example is given in FIG. 11 and a restriction map shown in FIG. 12. The Sox-9 gene sequence comprised bases 1265-27222 (gene bank accession number z46629)

### EXAMPLE 3

Experiments were conducted to illustrate the effectiveness of reducing oxygen tension as a means of differentiating MSSC cells towards NP cells.

MSSC cells were isolated and cultured as described in Example 1 at 1.1.1 and 1.1.2. These cells were then transformed with Sox-9 as described in Example 2 in order that expression of Sox 9 may be monitored as a marker of differentiation. Expression of aggrecan and Collagen type II was also monitored as a marker of differentiation.

FIG. 13 illustrates that MSSC cells cultured in monolayer in 1% oxygen for 2 weeks differentiated towards NP cells whereas cells cultured in monolayer in 20% oxygen for 2 weeks expressed negligible amounts of differentiation markers.

### EXAMPLE 4

The inventors conducted further development work whereby the differentiation steps described above (and particularly in Example 1 and 3) were combined.

The inventors found that using more than one differentiation technique could improve differentiation. For instance, MSSC cells encapsulated in alginate and loaded 3 times a week for 4 hours under a light exercise loading regime (0.8-1.7 MPa, 1 Hz) resulted in a doubling of total proteoglycan content of the alginate construct over 1 and 3 weeks in culture. Such cells had phenotypes closely resembling natural NP cells. FIG. 14 illustrates proteoglycan production (a differentiation markers) from such cells.

### EXAMPLE 5

The inventors developed a further IVD cell induction growth media comprising cartilage derived growth factor (CDMP) 1 and/or 2.

MSSC cells were cultured in alginate layer constructs, in standard media (DMEM/hams F12, 10% FCS, 100 U/ml Penicillin, 100 μg/ml Streptomycin, 250 ng/ml amphotericin, 2 mM glutamine and 50 μg/ml ascorbic acid). Cells were then treated with 1, 10 or 100 ng/ml CDMP 1 or CDMP 2. Following 48 hours of treatment alginate layer constructs were removed from culture, a papain digest performed and GAG content assessed using the DMMB assay. Concentration of GAG per alginate layer construct was then calculated.

The data illustrated in FIG. 15 demonstrates that CDMP treatment of MSSC cells increased their proteoglycan production (a marker of IVD cell differentiation). The effect seen was most optimum following treatment with 1 ng/ml CDMP 1 or 100 ng/ml CDMP 2.

### EXAMPLE 6

The inventors performed experiments to illustrate that co-culture of disc cells with MSSCs, which were in direct contact and at varying cell ratios in a standard media induces differentiation of MSSCs towards NP cells. It is worth noting that this co-culturing technique was performed in a media that did not incorporate growth factors such as TGFβ or CDMP.

Method:

Normal human NP cells and MSCs were cultured to confluence in monolayer, then MSCs were labelled with a green-fluorescent, cell permanent dye (CFDA). Cells were then seeded into the wells of a 24-well plate at the following ratios:

100% NP cells

100% MSCs

75% NP cells: 25% MSCs

50% NP cells: 50% MSCs

25% NP cells: 75% MSCs

At 7 and 14 days timepoints, MSCs and NP cells were separated by FACS utilising the difference in fluorescence between CFDA-labelled MSCs and unlabelled NP cells.

Following cell sorting, RNA was extracted using the TRIzol method and real-time PCR conducted using the SYBR-green method. Two house-keeping genes (18s and GAPDH) were used and their expression levels averaged. Target genes (type II collagen and aggrecan—i.e. markers of IVD cells) were analysed and their expression normalised to the averaged house-keeping gene expression and expression of the relevant target gene in control cells (also normalised to their own averaged house-keeping gene expression) using the 2^{-ΔΔCT} method.

Results:

Data was generated illustrating that expression of type II collagen mRNA expression after 7 days (A) and 14 days (B) of co-culture and aggrecan mRNA following 7 days (C) and 14 days (D) of co-culture. This data indicated that co-culture techniques promote differentiation towards IVD cells.

1. An isolated mesenchymal stromal stem cell (MSSC) that has been differentiated in vitro towards, or to, an intervertebral disc (IVD) cell phenotype.

2. An isolated mesenchymal stromal stem cell (MSSC) characterised in that it is:
a) differentiated in vitro towards, or to, a intervertebral disc (IVD) cell phenotype; and
b) genetically transformed with an exogenous gene which codes for a protein that reduces degeneration of an intervertebral disc.

3. The isolated mesenchymal stromal stem cell according to claim 1 wherein the cell produces an extracellular matrix.

4. The isolated mesenchymal stromal stem cell according to claim 3 wherein the extracellular matrix is identifiable as an IVD extracellular matrix and is distinguishable from an extracellular matrix produced by a chondrocyte.

5. The isolated mesenchymal stromal stem cell according to claim 4 wherein the IVD matrix is characterised by at least one of:
(a) aggrecan gene expression is greater than collagen type II gene expression;
(b) the proteoglycan versican is expressed; or
(c) the GAG: hydroxproline ratio is greater than 10:1.

6. The isolated mesenchymal stromal stem cell according to claim 1 wherein the cell is derived from blood, bone marrow, or adipose tissue.

7. The isolated mesenchymal stromal stem cell according to claim 6, wherein the cell is derived from bone marrow in the sternum, femur or iliac crest.

8. The isolated mesenchymal stromal stem cell according to claim 1, wherein the MSSC is differentiated using at least of the steps of:
(a) growth in a IVD cell induction medium containing TGFβ, CDMP1 or CDMP2;
(b) encapsulation of the MSSC;
(c) application of Load to the MSSCs;
(d) Co-culture of the MSSC with Nucleus Pulposus cells/IVD cells;
(e) Culture of the MSSC in conditioned media in which IVD cells have previously been grown;
(f) Culture in low oxygen tensions; or
(g) Genetically transformed using a gene regulator of IVD cell differentiation.

9. The isolated mesenchymal stromal stem cell according to claim 8 wherein differentiation is effected by using any combination of steps (a), (b), (c), (d), (e), (f) and (g).

10. The isolated mesenchymal stromal stem cell according to claim 9 wherein the MSSC is differentiated by encapsulating the MSSC in a gel; and growing the encapsulated cell in a medium for up to 5 weeks during which time a cyclical load equivalent to that experienced in vivo is exerted using hydraulic or other methodology.

11. The isolated mesenchymal stromal stem cell according to claim 10 wherein the media is an induction medium according to claim 8(a).

12. The isolated mesenchymal stromal stem cell according to claim 10 wherein the media is a conditioned medium according to claim 8(e).

13. The isolated mesenchymal stromal stem cell according to claim 10 wherein the MSSC is co-cultured with cells according to claim 8(d).

14. The isolated mesenchymal stromal stem cell according to claim 11, wherein the oxygen pressure is reduced to less than 5% of the atmosphere in which the cell is cultured.

15. The isolated mesenchymal stromal stem cell according to claim 2, wherein the exogenous gene is selected from the group consisting of genes encoding proteins involved in the regulation of inflammation, genes encoding cytokines, genes encoding inhibitors of cytokines, and genes encoding inhibitors of degradative enzymes.

16. The isolated mesenchymal stromal stem cell according to claim 2, wherein the exogenous gene encodes Interleukin 1 Receptor Antagonist (IL-1RA).

17. A composition for the treatment of spinal conditions characterized by degeneration of the intervertebral disc, comprising the mesenchymal stromal stem cell of claim 1.

18. The composition according to claim 17 wherein the spinal condition is Low Back Pain, degeneration of the intervertebral disc, age-related changes of the intervertebral disc or spondylolysis.

19. The composition according to claim 17 wherein the composition is configured for direct injection into an IVD exhibiting DIVD.

20. The composition according to claim 17 wherein the composition is configured for seeding onto or into biomaterial scaffolds or gels.

21. A method of treating spinal conditions characterized by degeneration of the intervertebral disc comprising:
providing a composition comprising an isolated MSSC that has been differentiated in vitro towards, or to, an IVD cell phenotype; and
administering said composition to a diseased intervertebral disc of a subject in need of such treatment.

22. A method of treating spinal conditions characterized by degeneration of the intervertebral disc comprising:
providing a composition comprising an isolated MSSC, wherein said MSSC has been:
(a) differentiated in vitro towards, or to, a IVD cell phenotype, and
(b) genetically transformed with an exogenous gene which codes for a
protein that reduces degeneration of an intervertebral disc; and
administering said composition to a diseased intervertebral disc of a subject in need of such treatment.

23. A method for causing mesenchymal stromal stem cells to differentiate towards IVD cells comprising exposing cultured mesenchymal stromal stem cells to increasing pressures of up to 30 psi (2.1 MPa).

24. A method for causing mesenchymal stromal stem cells to differentiate towards IVD cells comprising co-culturing NP cells and mesenchymal stromal stem cells (MSSCs) together.

25. A method for causing mesenchymal stromal stem cells to differentiate towards IVD cells comprising culturing mesenchymal stromal stem cells in media that has previously been exposed to NP cells.

26. A method for causing mesenchymal stromal stem cells to differentiate towards IVD cells comprising culturing mesenchymal stromal stem cells in an atmosphere in which oxygen pressure is reduced to less than 5%.

27. A method for causing mesenchymal stromal stem cells (MSSCs) to differentiate towards IVD cells comprising encapsulating MSSCs in a gel and growing the encapsulated cells in a medium for up to 5 weeks during which time a cyclical load equivalent to that experienced in vivo is exerted using hydraulic or other methodology

28. The method according to claim 27 wherein the media is an induction medium containing TGFβ, CDMP1 or CDMP2.

29. The method according to claim 27 wherein the media is a conditioned medium in which IVD cells have previously been grown.

30. The method according to claim 27 wherein the MSSCs are co-cultured with Nucleus Pulposus cells/IVD cells.

31. The method according to claim 27 wherein the oxygen pressure is reduced to less than 5% of the atmosphere in which the cells are cultured.

32. The method according to claim 28 wherein the oxygen pressure is reduced to less than 5% of the atmosphere in which the cells are cultured.

33. The method according to claim 29 wherein the oxygen pressure is reduced to less than 5% of the atmosphere in which the cells are cultured.

34. The method according to claim 30 wherein the oxygen pressure is reduced to less than 5% of the atmosphere in which the cells are cultured.

35. The isolated mesenchymal stromal stem cell according to claim 2 wherein the cell produces an extracellular matrix.

36. The isolated mesenchymal stromal stem cell according to claim 12, wherein the oxygen pressure is reduced to less than 5% of the atmosphere in which the cell is cultured.

37. The isolated mesenchymal stromal stem cell according to claim 13, wherein the oxygen pressure is reduced to less than 5% of the atmosphere in which the cell is cultured.


SEQUENCE LISTING (1 sequence)

SEQ ID NO: 1 - DNA, 5733 bp, Homo sapiens
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggactcgagtgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtaagcttgatcttggtggcgtgaaactcccgcacctcttcggccagcgccttgtagaagcgcgtatgggatctgactacaaagacgatgacgacaagggatcctccgaggactccgcgggctcgccctgcccgtcgggctccggctcggacaccgagaacacgcggccccaggagaacacgttccccaagggcgagcccgatctgaagaaggagagcgaggaggacaagttccccgtgtgcatccgcgaggcggtcagccaggtgctcaaaggctacgactggacgctggtgcccatgccggtgcgcgtcaacggctccagcaagaacaagccgcacgtcaagcggcccatgaacgccttcatggtgtgggcgcaggcggcgcgcaggaagctcgcggaccagtacccgcacttgcacaacgccgagctcagcaagacgctgggcaagctctggagacttctgaacgagagcgagaagcggcccttcgtggaggaggcggagcggctgcgcgtgcagcacaagaaggaccacccggattacaagtaccagccgcggcggaggaagtcggtgaagaacgggcaggcggaggcagaggaggccacggagcagacgcacatctcccccaacgccatcttcaaggcgctgcaggccgactcaagcttgccacactcctcctccggcatgagcgaggtgcactcccccggcgagcactcggggcaatcccagggcccaccgaccccacccaccacccccaaaaccgacgtgcagccgggcaaggctgacctgaagcgagaggggcgccccttgccagaggggggcagacagccccctatcgacttccgcgacgtggacatcggcgagctgagcagcgacgtcatctccaacatcgagaccttcgatgtcaacgagtttgaccagtacctgccgcccaacggccacccgggggtgccggccacgcacggccaggtcacctacacgggcagctacggcatcagcagcaccgcggccaccccggcgagcgcgggccacgtgtggatgtccaagcagcaggcgccgccgccacccccgcagcagcccccacaggccccgccggccccgcaggcgcccccgcagccgcaggcggcgcccccacagcagccggcggcacccccgcagcagccacaggcgcacacgctgaccacgctgagcagcgagccgggccagtcccagcgaacgcacatcaagacggagcagctgagccccagccactacagcgagcagcagcagcactcgccccaacagatcgcctacagccccttcaacctcccacactacagcccctcctacccgcccatcacccgctcacagtacgactacaccgaccaccagaactccagctcctactacagccacgcggcaggccagggcaccggcctctactccaccttcacctacatgaaccccgctcagcgccccatgtacacccccatcgccgacacctctggggtcccttccatcccgcagacccacagcccccagcactgggaacaacccgtctacacacagctcactcgaccttgagaattctttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcatcgatccataacttcgtataatgtatgctatacgaagttatccagatctggttctatagtgtcacctaaatcgtatgtgtatgatacataaggttatgtattaattgtagccgcgttctaacgacaatatgtccatagggcccctacgtcacccgccccgttcccacgccccgcgccacgtcacaaactccaccccctcattatcatattggcttcaatccaaaataaggtatattattgatgatggccgcagcggcccctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggggccgctgcggc